(19) United States
(12) Patent Application Publication
(10) Pub. No.: US 2014/0309674 A1
(43) Pub. Date: Oct. 16, 2014

(54) LIQUID EJECTION DEVICE AND MEDICAL APPARATUS

(71) Applicant: SEIKO EPSON CORPORATION, Tokyo (JP)

(72) Inventors: Hirokazu SEKINO, Chino-shi (JP); Hideki KOJIMA, Matsumoto-shi (JP); Takeshi SETO, Chofu-shi (JP)

(73) Assignee: SEIKO EPSON CORPORATION, Tokyo (JP)

(21) Appl. No.: 14/250,963

(22) Filed: Apr. 11, 2014

(30) Foreign Application Priority Data
Apr. 12, 2013 (JP) 2013-083609

(51) Int. Cl. A61B 17/3203

(52) U.S. Cl. CPC A61B 17/3203; A61B 2017/32032; A61B 2017/00539
USPC 606/167; 222/394

(57) ABSTRACT

A liquid ejection device that ejects a liquid from an ejection opening includes: an actuator that is stored in a storage container and pressurizes the liquid supplied from outside; an inlet channel that has a curved shape and is connected to a first surface of the actuator, and supplies the liquid to the actuator; and an outlet channel that is connected to the first surface of the actuator and causes the liquid to flow out from the actuator to the ejection opening.

This application claims the benefit of Japanese Patent Application No. 2013-83609, filed on Apr. 12, 2013. The content of the aforementioned application is incorporated herein by reference in its entirety.

### BACKGROUND

1. Technical Field

The present invention relates to technology of a liquid ejection device that ejects a liquid and a medical apparatus that uses the liquid ejection device.

2. Related Art

For example, technology regarding a liquid ejection device that ejects a liquid is disclosed in JP-A-2008-82202. The liquid ejection device disclosed in JP-A-2008-82202 describes technology in which excised matter that is excised by the liquid that is ejected from an ejection channel is removed by sucking in a suction channel.

The liquid ejection device disclosed in JP-A-2008-82202 has an excellent structure in that an actuator applies a pulse flow to the liquid supplied from an inlet channel and the liquid flows out from an outlet channel, and a pulsed liquid is ejected from an ejection opening.

Miniaturization (slimming) of the structure may be further pursued while taking advantage of such a structure. Furthermore, simplification of a structure of the inlet channel and reduction of a channel resistance may be pursued. Moreover, in the liquid ejection device, low cost, resource saving, ease of manufacturing or improvement of usability is desired.

### SUMMARY

An advantage of some aspects of the invention is to solve at least a part of the problems described above, and the invention can be implemented as the following aspects.

(1) An aspect of the invention provides a liquid ejection device that ejects a liquid from an ejection opening. The liquid ejection device includes: an actuator that is stored in a storage container and pressurizes the liquid supplied from outside; an inlet channel that has a curved shape and is connected to a first surface of the actuator, and supplies the liquid to the actuator; and an outlet channel that is connected to the first surface of the actuator and causes the liquid to flow out from the actuator to the ejection opening. According to the liquid ejection device with this configuration, since the inlet channel has the curved shape and is connected to the first surface of the actuator, it is possible to reduce a combined width of the actuator and the inlet channel.

(2) In the liquid ejection device according to the aspect described above, the inlet channel may be connected to a position that is shifted from a surface center in the first surface. In this case, it is possible for the inlet channel to be connected to the first surface by ensuring separation from the outlet channel.

(3) In the liquid ejection device according to the aspect described above, the outlet channel may be connected to a position that is shifted from the surface center in the first surface. According to the liquid ejection device with this configuration, it is possible for the outlet channel to be connected to the first surface by ensuring separation from the inlet channel.

(4) In the liquid ejection device according to the aspect described above, the inlet channel may be curved in a U shape by intersecting the outlet channel when viewed from a direction perpendicular to the normal of the first surface. According to the liquid ejection device with this configuration, it is possible to reduce a curvature of the U shape pipe path of the inlet channel.

(5) In the liquid ejection device according to the aspect described above, the liquid ejection device may further include: a vertical direction defining section that defines a vertical direction of the storage container, in which the outlet channel may be connected to a position that is further upward on the first surface than the inlet channel. According to the liquid ejection device with this configuration, since the outlet channel is connected to a position that is further upward on the first surface than the inlet channel, based on the vertical direction, it is possible to easily flow out air, which is mixed into the liquid flowed into from the inlet channel, from the outlet channel.

The vertical direction defining section may define the vertical direction as a usage direction of the storage container and may define the vertical direction of the storage container, based on the direction of the storage container in which functions and characteristics of the liquid ejection device are suitably exerted if the functions and characteristics are changed by the direction of the storage container. The vertical direction defining section may be a device that informs a user of information defining the vertical direction of the storage container through at least one of five senses such as a sense of sight, a sense of touch and a sense of hearing of the user using the liquid ejection device. The vertical direction defining section may include a device that draws attraction of the user when using the storage container to the vertical direction of the storage container from various elements such as shape, pattern, color, line images, characters, marks, sound, light, operability and design of the configurations provided in the liquid ejection device.

(6) In the liquid ejection device according to the aspect described above, the inlet channel may be made of a metal member. According to the liquid ejection device with this configuration, it is possible to suppress narrowing of an inner diameter in a portion in which the inlet channel is curved.

(7) Another aspect of the invention provides a medical apparatus using the liquid ejection device according to the aspect described above. According to the medical apparatus with this configuration, it is possible to reduce the combined width of the actuator and the inlet channel when viewed from an axial direction of the storage container.

Not all of a plurality of configuration elements included in each aspect of the invention described above are essential. In order to solve a part of or all of the problems described above, or in order to achieve a part of or all of the effects described in the specification, a configuration element that is a part of the plurality of configuration elements described above may be appropriately changed, deleted, and replaced with other, new configuration elements, and partial deletion of a limited content may be performed. Further, in order to solve a part of or all of the problems described above, or in order to achieve a part of or all of the effects described in the specification, an independent aspect of the invention may be achieved by combining a part of or all of the technical characteristics that are included in other aspects of the invention described above with a part of or all of the technical characteristics that are included in another aspect of the invention described above.

For example, an aspect of the invention may be implemented as a device that includes one or more elements among four elements of the storage container, the actuator, the inlet channel and the outlet channel. That is, the device may have or may not have the storage container. Further, the device may have or may not have the actuator. Further, the device may have or may not have the inlet channel. Further, the device may have or may not have the outlet channel. For example, the actuator may be configured as an actuator that is stored in the storage container and pressurizes the liquid supplied from outside. The inlet channel may be configured as an inlet channel that has a curved shape, is connected to the first surface of the actuator and supplies the liquid to the actuator. For example, the outlet channel may be configured as an outlet channel that is connected to the first surface of the actuator and causes the liquid to flow out from the actuator to the ejection opening. For example, such a device can be implemented as the liquid ejection device and can be also implemented as any device other than the liquid ejection device. According to the aspect described above, it is possible achieve at least one of small size, low cost, resource saving, ease of manufacturing, improvement of usability and the like. A part or all of the technical characteristics of each aspect of the liquid ejection device described above may be applied to the device.

The invention can be implemented in various aspects other than the device. For example, the invention can be implemented in an aspect of a method for ejecting the liquid or a method for manufacturing the liquid ejection device.

### BRIEF DESCRIPTION OF THE DRAWINGS

The invention will be described with reference to the accompanying drawings, wherein like numbers reference like elements.

FIG. 1 is an explanatory view illustrating a configuration of a liquid ejection device.

FIG. 2 is an explanatory view illustrating an internal structure of a handpiece.

FIG. 3 is an explanatory view illustrating extension aspects of an inlet channel and an outlet channel.

FIG. 4 is an explanatory view illustrating an extension aspect 1.

FIG. 5 is an explanatory view illustrating an extension aspect 2.

FIG. 6 is an explanatory view illustrating an extension aspect 3.

FIG. 7 is an explanatory view illustrating an extension aspect 4.

FIG. 8 is an explanatory view illustrating a handpiece including a grip.

FIG. 9 is an explanatory view illustrating a handpiece in which a curved section is formed in an ejection pipe.

### DESCRIPTION OF EXEMPLARY EMBODIMENTS

### A. First Embodiment

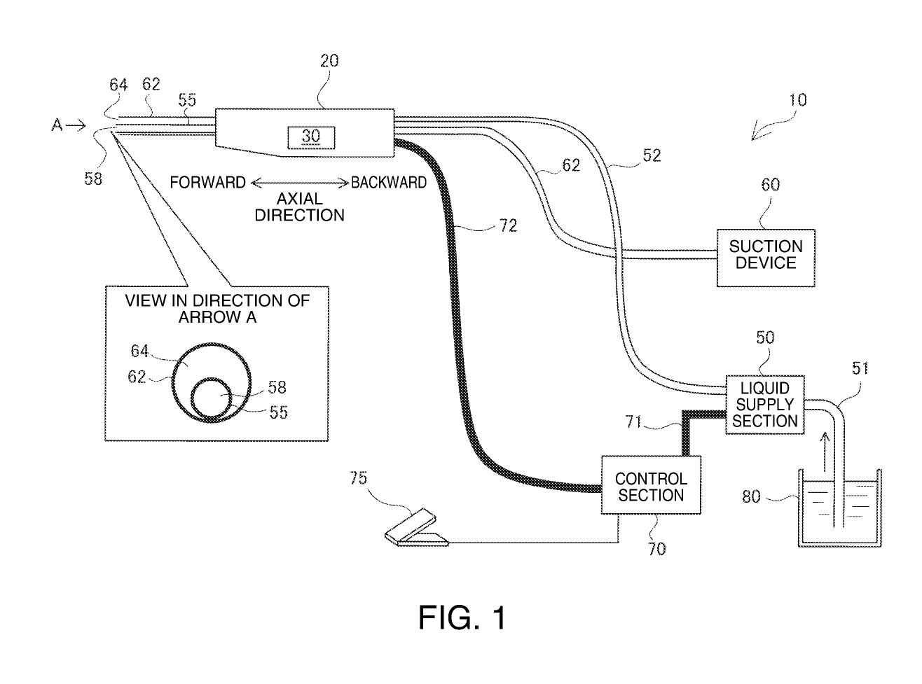

FIG. 1 is an explanatory view describing a configuration of a liquid ejection device 10 as a first embodiment of the invention. The liquid ejection device 10 of the embodiment is a medical apparatus used in medical institutions and has a function as a scalpel for performing incision on or excision of a diseased part by ejecting the liquid with respect to the diseased part.

The liquid ejection device 10 includes a handpiece 20 as a storage container, a liquid supply section 50, a suction device 60 and a control section 70, and is connected to a liquid container 80. The liquid supply section 50 and the liquid container 80 are connected by a connection tube 51. The liquid supply section 50 and the handpiece 20 are connected by a liquid supply channel 52. In the embodiment, the connection tube 51 and the liquid supply channel 52 are formed of a resin.

The liquid container 80 accommodates physiological saline solution as the liquid. The liquid supply section 50 supplies the liquid that is sucked from the liquid container 80 through the connection tube 51 to the handpiece 20 through the liquid supply channel 52. Moreover, in the embodiment, the physiological saline solution is employed as the liquid, but the invention is not limited to the embodiment and it is possible to employ various types of liquids such as sterile water or pure water.

The handpiece 20 is a device that is grasped and operated by a user of the liquid ejection device 10. The handpiece 20 includes an actuator 30, an ejection pipe 55, an ejection opening 58, and a suction force adjustment mechanism 65. In the embodiment, a direction along the ejection pipe 55 of the handpiece 20 is referred to as an axial direction. A direction in the axial direction, which includes the ejection pipe 55 is referred to as a forward direction. Pulsation having a predetermined frequency is applied to the liquid, which is supplied from the liquid supply section 50 to the handpiece 20 through the liquid supply channel 52, by the actuator 30, and the liquid is supplied to the ejection pipe 55. The liquid supplied to the ejection pipe 55 is ejected from the ejection opening 58 as a pulsed liquid. The user performs incision on or excision of a diseased part by applying the pulsed liquid ejected from the ejection opening 58 to the diseased part of the patient. In the embodiment, the ejection pipe 55 is formed of stainless steel. However, the ejection pipe 55 may be formed of a material having a predetermined rigidity or higher such as other metals, for example, brass or the like, or reinforced plastic. The suction force adjustment mechanism 65 is a mechanism capable of allowing adjusting a degree of the suction of the suction device 60 by the user. In the embodiment, as the degree of the suction, a suction amount per unit time is employed.

The control section 70 transmits a drive signal to the actuator 30 through a signal cable 72 and controls the liquid supply section 50 through a control cable 71, thereby performing control of a flow amount of the liquid that is supplied to the actuator 30. A foot switch 75 that is operated by a foot of the user is connected to the control section 70. When the user turns on the foot switch 75, the control section 70 controls the liquid supply section 50 and performs the supply of the liquid to the actuator 30, and transmits the drive signal to the actuator 30. Therefore, the pulsation is applied to the liquid that is supplied to the actuator 30 and the pulsed liquid is ejected from the ejection opening 58. Moreover, the expression “the liquid is ejected in a pulsed manner” means that the liquid is ejected in a state accompanied with changes in the flow amount or a flow speed of the liquid that is ejected. Intermittent ejection in which the liquid is ejected by repeating the ejection and stop of the liquid is included in the form in which the liquid is ejected in the pulsed manner. However, as long as the flow amount or the flow speed of the liquid changes, it is not necessary for the form to be the intermittent ejection.

The suction device 60 is a device that sucks the liquid, excised matter or the like (hereinafter, also referred to as suction matter) around the ejection opening 58. The suction device 60 and the handpiece 20 are connected by a suction channel 62. The suction channel 62 extends near a leading end of the ejection pipe 55 through an inside the handpiece 20. The ejection pipe 55 is inserted into the inside of the suction channel 62. As illustrated in a direction of arrow A in FIG. 1, a channel (hereinafter, also referred to as a gap channel) in which the liquid, which is sucked from a suction port 64 that is the leading end of the suction channel 62, flows, is formed between an outer wall of the ejection pipe 55 and an inner wall of the suction channel 62. The liquid that flows from the suction port 64 into the gap channel is sucked into the suction device 60 through the suction channel 62. The suction amount per unit time that is sucked from the suction port 64 is capable of being adjusted by the user operating the suction force adjustment mechanism 65.

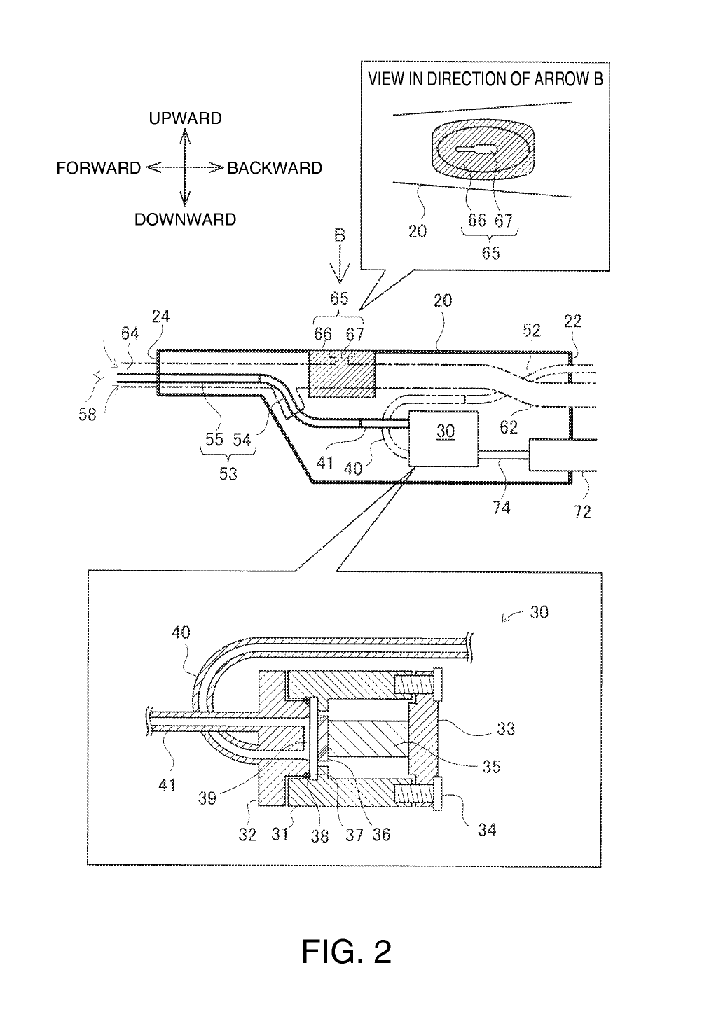

FIG. 2 is an explanatory view describing an internal structure of the handpiece 20. First, a structure of the actuator 30 is described. As illustrated in a lower section of FIG. 2, the actuator 30 includes a first case 31, a second case 32, a third case 33, bolts 34, a piezoelectric element 35, a reinforced plate 36, a diaphragm 37, a gasket 38, an inlet channel 40, and an outlet channel 41. The first case 31 and the second case 32 are bonded facing each other. The first case 31 is a cylindrical member. One end section of the first case 31 is closed by fixing the third case 33 with the bolts 34. The piezoelectric element 35 is disposed in a space that is formed inside the first case 31.

The piezoelectric element 35 is a multilayered piezoelectric element. One end section of the piezoelectric element 35 is fixed to the diaphragm 37 through the reinforced plate 36. The other end section of the piezoelectric element 35 is fixed to the third case 33. The diaphragm 37 is formed of a metal thin film and a peripheral section thereof is fixed to the first case 31. A liquid chamber 39 is formed between the diaphragm 37 and the second case 32. The volume of the liquid chamber 39 is changed by driving of the piezoelectric element 35.

The second case 32 includes the inlet channel 40 into which the liquid flows and the outlet channel 41 from which the liquid flows out. The inlet channel 40 and the outlet channel 41 are connected to and extend from a forward end surface (a leading end surface) of the actuator 30, that is, a leading end surface of the second case 32, in predetermined forms. The extending forms of the inlet channel 40 and the outlet channel 41 from the second case 32 are described below in detail.

The liquid supply channel 52 is connected to the inlet channel 40. The ejection pipe 55 is connected to the outlet channel 41 through a connection tube 54. A channel that is formed of two pipe paths of the connection tube 54 and the ejection pipe 55 is also referred to as an ejection channel 53. The liquid supplied from the liquid supply section 50 is supplied to the liquid chamber 39 through the liquid supply channel 52. When the piezoelectric element 35 vibrates with a predetermined frequency, the volume of the liquid chamber 39 is changed through the diaphragm 37 and the liquid that is accommodated is pressurized. The liquid that is pressurized is discharged from the ejection opening 58 through the outlet channel 41, the connection tube 54 and the ejection pipe 55.

The suction channel 62 communicates with the leading end section 24 from a back end section 22 of the handpiece 20. On the inside of the handpiece 20, the ejection channel 53 is inserted into the suction channel 62. The suction channel 62 is led out from the leading end section 24 of the handpiece 20 together with the ejection pipe 55.

The ejection channel 53 and the suction channel 62 are separated on the inside of the handpiece 20. The ejection channel 53 is separated from the suction channel 62 by being curved in a separation portion of the two channels. The ejection channel 53 is gently curved and the curvature is preferably decreased in the separation portion. The suction channel 62 is arranged substantially linearly in the separation portion. Moreover, in the embodiment, an aspect in which the suction channel 62 is arranged substantially linearly in the separation portion is employed, but the suction channel 62 may be arranged in a curved shape having a curvature less than that of the curve of the ejection channel in the separation portion.

The connection tube 54 among the pipe paths configuring the ejection channel 53 is disposed substantially parallel to the axial direction of the handpiece 20 that has a columnar shape and is connected to the outlet channel 41 after being separated from the suction channel 62. Further, in the embodiment, the connection tube 54 after being separated is disposed substantially parallel to the suction channel 62.

The suction force that sucks the liquid or the like from the suction port 64 by the suction channel 62 is capable of being adjusted in the suction device 60 and is capable of being adjusted by the user operating an operation section 66 of the suction force adjustment mechanism 65 included in the handpiece 20.

The suction force adjustment mechanism 65 includes the operation section 66 and a suction adjustment hole 67. The suction force adjustment mechanism 65 is a member that is formed of a resin. A channel configuring a part of the suction channel 62 is formed inside the suction force adjustment mechanism 65. Practically, the suction channel 62 is connected to both ends of the channel formed in the suction force adjustment mechanism 65. The operation section 66 is a portion in the suction force adjustment mechanism 65 which is exposed to the outside of the handpiece 20 and is a portion that is operated with a finger of the user.

The suction adjustment hole 67 communicates with the suction channel 62 and the operation section 66. As illustrated in a direction of arrow B in FIG. 2, an opening section of the suction adjustment hole 67 is formed in the operation section 66. The user opens and closes the suction adjustment hole 67 with a finger when grasping the handpiece 20. The suction force adjustment mechanism 65 adjusts the flow amount of air flowing from the outside into the suction channel 62 through the suction adjustment hole 67 and adjusts the pressure (hereinafter, also referred to as a suction pressure) inside the suction channel 62, depending on a size of a surface area of the suction adjustment hole 67 that is closed by the user. That is, the suction force adjustment mechanism 65 adjusts the suction amount per unit time.

The suction adjustment hole 67 is provided on the suction channel 62 after the ejection pipe 55 separates. In other words, the suction adjustment hole 67 is provided on the suction channel 62 on the back end side with respect to the separation portion of the ejection pipe 55 and the suction channel 62.

If the suction force adjustment mechanism 65 is directed upward with respect to gravity, the suction matter is extremely difficult to be sucked from the suction adjustment hole 67 to the outside and then the function thereof is preferably exerted. When grasping the handpiece 20, the user adjusts the suction amount per unit time of the suction device 60 by directing the suction force adjustment mechanism 65 upward and by pressing the suction adjustment hole 67 included in the operation section 66 downward with a finger. Moreover, when grasping the suction force adjustment mechanism 65 in the upward direction with respect to gravity, a position of each configuration of the liquid ejection device 10 is determined so that the function of the suction force adjustment mechanism 65 or operability of the user is suitable, but the suction force adjustment mechanism 65 does not force the user to necessarily use the handpiece 20 in the upward direction. Hereinafter, in the handpiece 20, the direction of the suction force adjustment mechanism 65 is defined as the upward direction.

The signal cable 72 is inserted from the back end section 22 of the handpiece 20. Two electrode wires 74 consisting of plus and minus inserted into the signal cable 72 are connected to the piezoelectric element 35 inside the actuator 30. The drive signal transmitted from the control section 70 is transmitted to the piezoelectric element 35 through the electrode wires 74 inside the signal cable 72. The piezoelectric element 35 expands and contracts, based on the drive signal.

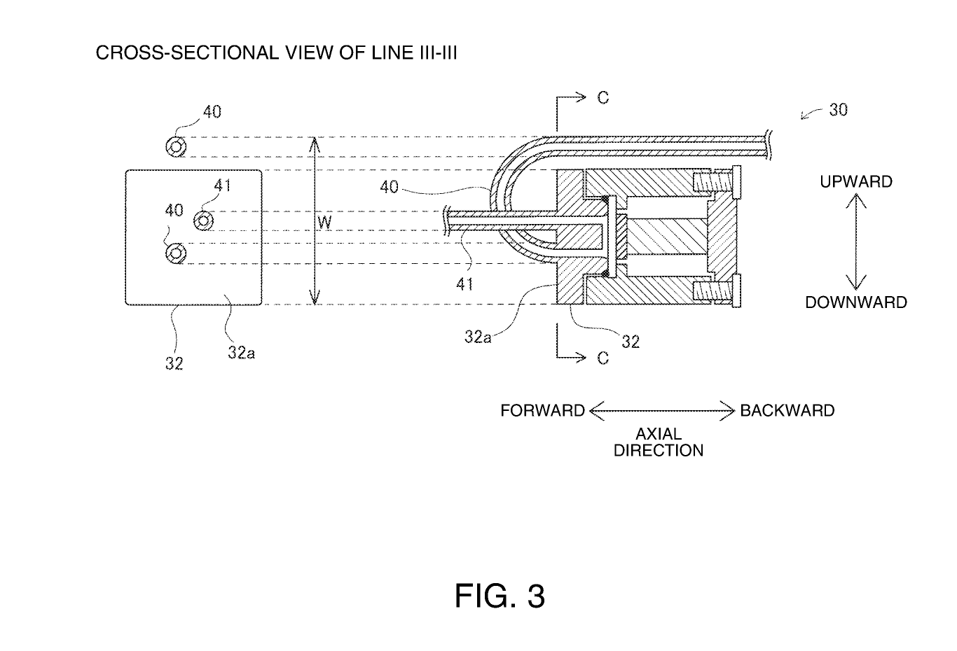

Next, the extending forms of the inlet channel 40 and the outlet channel 41 are described. FIG. 3 is an explanatory view describing the extending forms of the inlet channel 40 and the outlet channel 41 from the second case 32. As illustrated in FIG. 3, the outlet channel 41 extends from the second case 32 in the forward direction of the axial direction of the handpiece 20. The inlet channel 40 extends from the second case 32, is bent in a curved shape and is stretched toward the back end section 22 (a backward direction in FIG. 3) of the handpiece 20. In the embodiment, particularly, the inlet channel 40 is bent in a U shape.

In the embodiment, the inlet channel 40 and the outlet channel 41 are stainless steel pipes and are connected to the second case 32 by welding. The inlet channel 40 and the outlet channel 41 are not limited to stainless steel and may employ other metal pipes such as brass or aluminum. As illustrated in a cross-sectional view of line C-C of the actuator 30, positions in which the inlet channel 40 and the outlet channel 41 are connected to the second case 32 are positions which are shifted from a surface center of the forward end surface (hereinafter, also referred to as a leading end surface 32a) of the second case 32. That is, the inlet channel 40 and the outlet channel 41 are connected at positions away from the surface center of the substantially rectangular leading end surface 32a. Moreover, in the embodiment, the leading end surface 32a is the substantially rectangular shape, but as the shape of the leading end surface 32a, various shapes such as a circular shape, a triangular shape and other polygonal shapes may be employed. For example, in a case of the triangular shape, the inlet channel 40 and the outlet channel 41 may be connected to positions away from a weight center of the triangular leading end surface 32a. As the surface center of the leading end surface 32a, it is possible to employ a unique point of the figure such as the center and the weight center of the leading end surface 32a.

When a direction in which the suction force adjustment mechanism 65 is provided in the handpiece 20 is assumed as the upward direction, the outlet channel 41 extends by being connected to the leading end surface 32a at a position that is upward from the surface center of the leading end surface 32a of the second case 32. The inlet channel 40 extends by being connected to the leading end surface 32a at a position that is downward from the surface center of the leading end surface 32a of the second case 32 and is bent in a U shape in the upward direction, and stretches toward the backward direction in the axial direction intersecting the outlet channel 41.

When the inlet channel 40 and the outlet channel 41 are disposed as described above, the liquid entering the liquid chamber 39 from the inlet channel 40 flows out from the outlet channel 41 by flowing in the upward direction. Further, for example, even if air is present in the liquid chamber 39, air moves in the upward direction by buoyancy and flows out from the outlet channel 41.

As described above, since the inlet channel 40 has the curved shape (in the embodiment, particularly, the U shape) and is connected to the leading end surface 32a, it is possible for a combined width (a width W in FIG. 3) of the actuator 30 and the inlet channel 40 viewed from the axial direction (the front of the cross section of line C-C) of the handpiece 20 to be thin. As a result, it is possible for the handpiece 20 storing the actuator 30 and the inlet channel 40 to be thin. Further, in the embodiment, the curved shape means that a connecting point between the inlet channel 40 and the liquid supply channel 52 inside the handpiece 20 and a connecting point between the inlet channel 40 and the actuator 30 are connected with an angle that is greater than 90 degrees and 270 degrees or less between the connecting point between the inlet channel 40 and the liquid supply channel 52 inside the handpiece 20, and the connecting point between the inlet channel 40 and the actuator 30. Further, a more desirable curved shape has an angle that is greater than 160 degrees and less than 200 degrees and then it is possible to achieve miniaturization of the handpiece 20.

Since the inlet channel 40 extends by being connected to the leading end surface 32a at the position that is downward from the surface center thereof and the outlet channel 41 extends by being connected to the leading end surface 32a at the position that is upward from the surface center thereof, the air entering the liquid chamber 39 together with the liquid having flowed in from the inlet channel 40 or air bubbles generated in the liquid chamber 39 move in the upward direction by buoyancy and are likely to flow out from the outlet channel 41. Therefore, the pressure applied to the liquid in the liquid chamber 39 by the piezoelectric element 35 is unlikely to be absorbed by the air or air bubbles mixed into the actuator 30 and it is possible to generate sufficient pulse flow by the piezoelectric element 35.

Since the inlet channel 40 and the outlet channel 41 are connected to and extend from the positions shifted from the surface center of the leading end surface 32a, it is possible to connect to the leading end surface 32a with a sufficient gap therebetween. Since the inlet channel 40 is connected to and extends from the leading end surface 32a of the second case 32 at the position that is downward from the surface center thereof and is curved in the U shape in the upward direction, and stretches toward the backward direction of the axial direction intersecting the outlet channel 41, it is possible to reduce a curvature of the U shape pipe path.

Since the inlet channel 40 is formed in a state of being connected to the leading end surface 32a, the structure of the inlet channel 40 is simple and it is possible to easily form the inlet channel 40, compared to a case where the inlet channel 40 is formed by connecting the pipe to another side surface of the second case 32 and by molding a part of the second case 32 (for example, JP-A-2008-82202). Further, in the embodiment, since the inlet channel 40 is curved in the U shape and an elbow that may cause a channel resistance is not present, it is possible to ensure a sufficient flow speed (flow amount).

Furthermore, since the inlet channel 40 is a pipe made of a metal (in the embodiment, stainless steel), an inner diameter of the pipe is unlikely to be collapsed even if the pipe is bent into the U shape and it is possible to ensure sufficient channel cross-sectional area.

As a corresponding relationship between the embodiment described above and the appended claims, the leading end surface 32a corresponds to a first surface described in the appended claims, the axis of the handpiece 20 corresponds to the normal to the first surface described in the appended claims, and the suction force adjustment mechanism 65 corresponds to a vertical direction defining section described in the appended claims.

### B. Modification Example

Moreover, the invention is not limited to the embodiment described above and it is possible to implement the invention in various aspects within the scope that does not depart from the spirit thereof and, for example, the following modifications are possible.

### B1 Modification Example 1

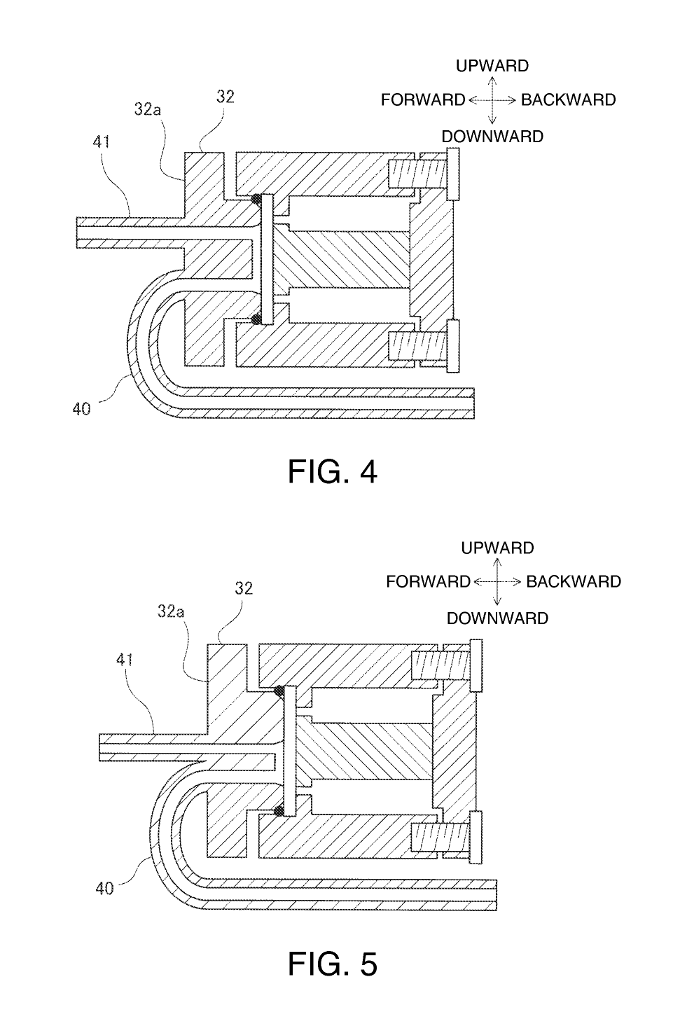

In the embodiment described above, the aspect is employed in which all of the inlet channel 40 and the outlet channel 41 are connected to and extend from the positions shifted from the surface center of the leading end surface 32a, but as the extension aspect of the inlet channel 40 and the outlet channel 41, it is possible to employ various extension aspects. FIGS. 4 to 7 are respective explanatory views illustrating an example of the inlet channel 40 and the outlet channel 41.

In the extension aspect (extension aspect 1) of FIG. 4, the outlet channel 41 extends from a position that is upward from the surface center of the leading end surface 32a and the inlet channel 40 extends from a position that is downward from the surface center of the leading end surface 32a and is bent in a U shape. As this case, it is possible to obtain the same advantage as that of the embodiment described above.

In an extension aspect (extension aspect 2) of FIG. 5, the outlet channel 41 extends from the surface center of the leading end surface 32a and the inlet channel 40 extends from a position that is downward from the surface center of the leading end surface 32a and is bent downward in a U shape. Also in this case, it is possible for a combined width of the actuator 30 and the inlet channel 40 to be thin. As a result, it is possible for the handpiece 20 storing the actuator 30 and the inlet channel 40 to be thin. Further, the air entering the liquid chamber 39 together with the liquid having flowed in from the inlet channel 40 moves upward by buoyancy and is likely to flow out from the outlet channel 41. Therefore, the pressure applied to the liquid in the liquid chamber 39 by the piezoelectric element 35 is unlikely to be absorbed by the air and it is possible to generate sufficient pulse flow by the piezoelectric element 35.

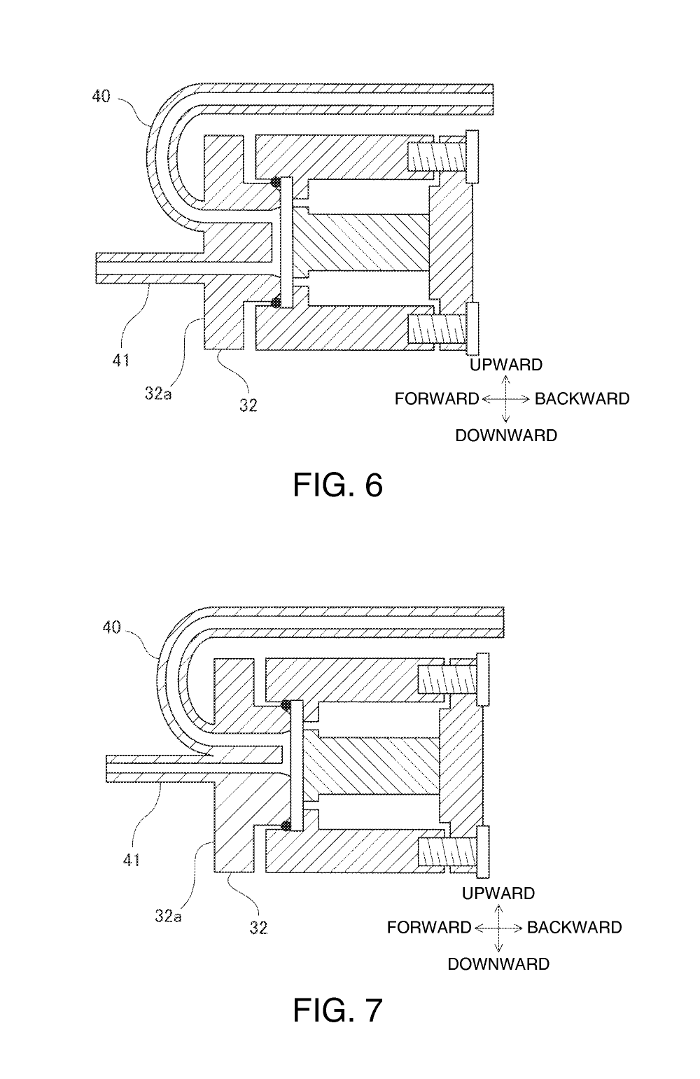

In an extension aspect (extension aspect 3) of FIG. 6, the outlet channel 41 extends from a position that is downward from the surface center of the leading end surface 32a and the inlet channel 40 extends from a position that is upward from the surface center of the leading end surface 32a and is bent upward in a U shape. Also in this case, it is possible for a combined width of the actuator 30 and the inlet channel 40 to be thin. Further, since the inlet channel 40 and the outlet channel 41 extend from the positions which are shifted from the surface center of the leading end surface 32a, it is possible to connect to the leading end surface 32a with a sufficient gap therebetween.

In an extension aspect (extension aspect 4) of FIG. 7, the outlet channel 41 extends from the surface center of the leading end surface 32a and the inlet channel 40 extends from a position that is upward from the surface center of the leading end surface 32a and is bent upward in a U shape. Also in this case, it is possible for a combined width of the actuator 30 and the inlet channel 40 to be thin.

As another extension aspect, it is possible to employ an aspect in which the inlet channel 40 extends from the surface center of the leading end surface 32a and is bent upward in a U shape and then stretches backward, and the outlet channel 41 extends from a position that is shifted from the surface center of the leading end surface 32a.

### B2 Modification Example 2

In the embodiment described above, the liquid ejection device 10 includes the suction force adjustment mechanism 65, but the invention may not include the suction force adjustment mechanism 65. Also in this case, it is possible to obtain the same advantage as that of the embodiment described above.

### B3 Modification Example 3

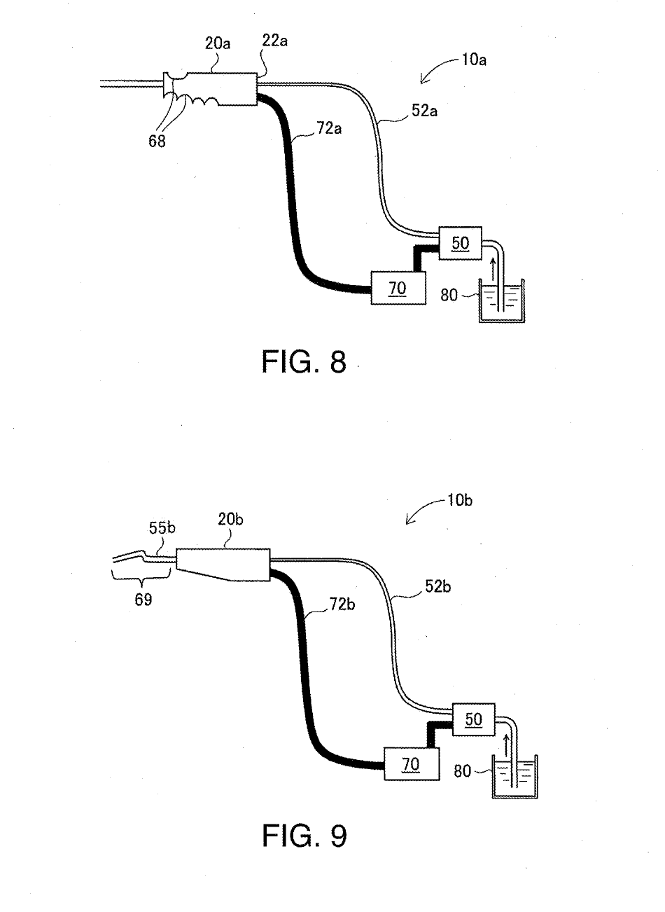

In the embodiment described above, as the vertical direction defining section, the suction force adjustment mechanism 65 is employed, but the invention is not limited to the embodiment and various configurations may be employed. FIG. 8 is an explanatory view illustrating a handpiece 20a including a grip 68 as the vertical direction defining section. The handpiece 20a defines the vertical direction by a shape of the grip 68. Further, FIG. 8 illustrates a liquid ejection device 10a that does not include the suction device 60 and the suction channel 62. As described above, even if the handpiece 20a does not include the suction force adjustment mechanism 65, it is possible to obtain the same advantage as that of the embodiment described above by providing the grip 68 as the vertical direction defining section.

FIG. 9 is an explanatory view illustrating a handpiece 20b in which a curved section 69 is formed in an ejection pipe 55b as the vertical direction defining section. A liquid ejection device 10b defines the vertical direction of the handpiece 20b by a shape of the curved section 69.

In addition, the vertical direction may be defined by a line image such as an arrow or characters illustrating the vertical direction of the handpiece. The vertical direction of the handpiece 20 may be defined by forming a cross sectional shape of the handpiece 20 in a columnar oval and by drawing the ejection pipe 55 out from an upper side from the center of the oval in the leading end section 24 of the handpiece 20. The user may be informed of the vertical direction defined in advance by providing a tilt sensor or a level in the handpiece 20. The user may be informed of the vertical direction by reading the direction of the handpiece 20 with a sensor provided outside the handpiece 20.

The vertical direction defining section may be a device that defines the vertical direction of the handpiece as a function of only the handpiece, may be a device that defines the vertical direction of the handpiece as a function of a device provided in the liquid ejection device that is provided as a separate body from the handpiece, and may be a device that defines the vertical direction of the liquid ejection device by cooperating with a device that is different from the liquid ejection device 10.

The suction force adjustment mechanism 65 of the first embodiment defines the vertical direction of the handpiece 20 as the direction in which operability of the user or the suitable functions of the suction force adjustment mechanism 65 is exerted. The grip 68 illustrated in FIG. 8 defines the vertical direction of the handpiece 20a by a sense of touch of the user. The curved section 69 illustrated in FIG. 9 defines the vertical direction of the handpiece 20b by a sense of sight of the user. As described above, the vertical direction defining section may define the vertical direction as a usage direction of the handpiece 20 when the user uses the handpiece 20, and may define the vertical direction of the handpiece 20, based on the direction of the handpiece 20 in which functions and characteristics of the handpiece 20 are suitably exerted if the functions and characteristics are changed by the direction of the handpiece 20. The vertical direction defining section may be a device that informs a user of information defining the vertical direction of the handpiece 20 through at least one of five senses such as the sense of sight, the sense of touch and the sense of hearing of the user using the liquid ejection device. The vertical direction defining section may be a device that draws attraction of the user when using the handpiece 20 to the vertical direction of the handpiece 20 from various elements such as shape, pattern, color, line images, characters, marks, sound, light, operability and design of the configurations provided in the liquid ejection device. Further, the vertical direction defining section may also be a device that prevents the user from using the liquid ejection device in a direction other than the vertical direction that is defined by the user in advance by various elements provided in the liquid ejection device.

### B4 Modification Example 4

In the embodiment described above, the liquid ejection device 10 is used as the medical apparatus. However, the liquid ejection device 10 may be used as an apparatus other than the medical apparatus. For example, the liquid ejection device 10 may be used as a cleaning apparatus for removing soiling on an object by applying the liquid that is ejected to the object, or a drawing apparatus that draws characters, graphics or the like by the liquid that is ejected. Also in this case, it is possible to obtain the same advantage as that of the embodiment described above.

### B5 Modification Example 5

In the embodiment described above, the physiological saline solution is employed as the liquid, but the invention is not limited to the embodiment and it is possible to employ various types of liquids such as sterile water or pure water.

### B6 Modification Example 6

In the embodiment described above, the inlet channel may be configured of one member or may be configured of a plurality of members or a plurality of channels. Further, the curved shape may be formed by combining the plurality of members or the plurality of channels.

### B7 Modification Example 7

In the embodiment described above, the leading end surface is employed as the first surface, but another surface provided in the actuator may be employed as the first surface. The leading end surface is preferable as the first surface.

What is claimed is:

1. A liquid ejection device that ejects a liquid from an ejection opening, comprising:
an actuator that is stored in a storage container and pressurizes the liquid supplied from outside;
an inlet channel that has a curved shape and is connected to a first surface of the actuator, and supplies the liquid to the actuator; and
an outlet channel that is connected to the first surface of the actuator and causes the liquid to flow out from the actuator to the ejection opening.

2. The liquid ejection device according to claim 1,
wherein the inlet channel is connected to a position that is shifted from a surface center in the first surface.

3. The liquid ejection device according to claim 1,
wherein the outlet channel is connected to a position that is shifted from a surface center in the first surface.

4. The liquid ejection device according to claim 2,
wherein the inlet channel is curved in a U shape by intersecting the outlet channel when viewed from a direction perpendicular to the normal of the first surface.

5. The liquid ejection device according to claim 1, further comprising:
a vertical direction defining section that defines a vertical direction of the storage container,
wherein the outlet channel is connected to a position that is further upward on the first surface than the inlet channel, based on the vertical direction.

6. The liquid ejection device according to claim 1,
wherein the inlet channel is made of a metal member.

7. A medical apparatus using the liquid ejection device according to claim 1.

8. A medical apparatus using the liquid ejection device according to claim 2.

9. A medical apparatus using the liquid ejection device according to claim 3.

10. A medical apparatus using the liquid ejection device according to claim 4.

11. A medical apparatus using the liquid ejection device according to claim 5.

12. A medical apparatus using the liquid ejection device according to claim 6.
